(19) United States
(12) Patent Application Publication
(10) Pub. No.: US 2018/0327519 A1
(43) Pub. Date: Nov. 15, 2018

(54) AMPHIPHILIC RAFT AGENT

(71) Applicant: Commonwealth Scientific and Industrial Research and Innovation Park, Action, Australian Capitol Territory (AU)

(72) Inventors: Craig L. Francis, Vermont, Victoria (AU); James Gardiner, Mulgrave, Victoria (AU); Ivan Martinez Botella, Aspendale, Victoria (AU); John Tsanaktsidis, Lysterfield, Victoria (AU)

(73) Assignee: Commonwealth Scientific and Industrial Research Organization, Acton, Australian Capital Territory (AU)

(21) Appl. No.: 15/755,842

(22) PCT Filed: Jul. 20, 2016
(86) PCT No.: PCT/AU2016/050643

§ 371 Date: Feb. 27, 2018

(30) Foreign Application Priority Data
Aug. 28, 2015 (AU) 2015903499

(51) Int. Cl. C08F 2/38; C07C 381/00

(52) U.S. Cl. CPC C08F 2/38; C08F 2438/03; C07C 381/00

(57) ABSTRACT

This invention relates to a RAFT agent formula (I).

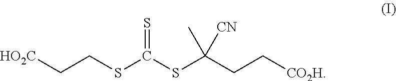 [SMILES: CC(C#N)(CCC(=O)O)SC(=S)SCCC(=O)O]

### FIELD OF THE INVENTION

The present invention relates generally to RAFT polymerisation. More specifically, the invention relates to a particular class of RAFT agent, a method of preparing the same, polymer prepared using the RAFT agent, and to a method of preparing polymer using the RAFT agent.

### BACKGROUND OF THE INVENTION

Reversible addition-fragmentation chain transfer (RAFT) polymerisation, as described in International Patent Publication No. WO 98/01478, is a polymerisation technique that exhibits the characteristics associated with living polymerisation. Living polymerisation is generally considered in the art to be a form of chain polymerisation in which irreversible chain termination is substantially absent. An important feature of living polymerisation is that polymer chains will continue to grow while monomer and the reaction conditions to support polymerisation are provided. Polymers prepared by RAFT polymerisation can advantageously exhibit a well defined molecular architecture, a predetermined molecular weight and a narrow molecular weight distribution or low dispersity (Ð).

RAFT polymerisation is believed to proceed under the control of a RAFT agent according to a mechanism which is simplistically illustrated below in Scheme 1.

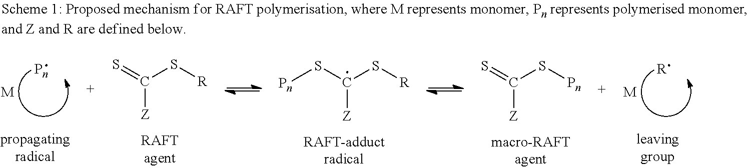

With reference to Scheme 1, R represents a group that functions as a free radical leaving group under the polymerisation conditions employed and yet, as a free radical leaving group, retains the ability to reinitiate polymerisation. Z represents a group that functions to convey suitable reactivity to the C═S moiety in the RAFT agent towards free radical addition without slowing the rate of fragmentation of the RAFT-adduct radical to the extent that polymerisation is unduly retarded. The ability for both R and Z to function in this way for a given agent is known to be influenced by the nature of the monomer to be polymerised and the polymerisation conditions.

Suitable R and Z groups of a RAFT agent for use in a given polymerisation reaction are typically selected having regard to the type of monomer that is to be polymerised.

In addition to selecting appropriate R and Z groups in the context of a monomer to be polymerised, the resulting RAFT agent must also have sufficient solubility in the reaction medium within which the polymerisation is to be conducted. For example, if the polymerisation is to be conducted in an aqueous reaction medium the selected RAFT agent will need to have sufficient solubility in that aqueous medium. Similarly, if the polymerisation is to be performed in an organic reaction medium, the RAFT agent must have sufficient solubility within that organic medium. In practice, balancing the requirements of suitable R and Z groups to provide for appropriate reactivity and solubility in a given reaction medium typically results in a need for using different RAFT agents when polymerisations are to be performed in either an aqueous or organic reaction media.

Although there are numerous advantages to be gained by using conventional RAFT agents to form polymer, it would nonetheless be desirable to provide a RAFT agent that offered further or new utility relative to those currently known.

### SUMMARY OF THE INVENTION

The present invention provides a RAFT agent of formula (I):

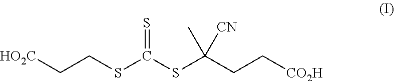 [SMILES: CC(C#N)(CCC(=O)O)SC(=S)SCCC(=O)O]

It has now been surprisingly found that a RAFT agent of formula (I) can advantageously be used to polymerise monomer in aqueous and organic reaction media. In other words, not only does the RAFT agent of formula (I) present R and Z groups that promote sufficient reactivity of the agent to control polymerisation of a diverse range of monomers, but the agent surprisingly has been found to exhibit unique solubility characteristics that enable monomer to be polymerised in either aqueous or organic reaction media.

The RAFT agent in accordance with the invention can be used to polymerise monomer in either aqueous or organic reaction media to afford polymer having a low dispersity (Ð). For example, polymer formed using a RAFT agent of formula (I) can be provided with a dispersity (Ð) of less than 1.5, or less than 1.4, or less than 1.3, or less than 1.2, or less than 1.1.

Without wishing to be limited by theory, it is believed the specific structural features of the RAFT agent of formula (I) provide for a unique solubility profile that enables the agent to have sufficient solubility in both aqueous and organic reaction media to effectively and efficiently control the polymerisation of a diverse range of monomers according to a RAFT mechanism.

In practice, those skilled in the art using conventional RAFT agents would typically employ different RAFT agents to control the polymerisation of monomer in aqueous and organic reaction media. The RAFT agent of formula (I) is therefore particularly versatile and simplifies the range of RAFT agents a person skilled in the art would typically need to have on hand to polymerise monomer in aqueous and organic reaction media.

The present invention therefore also provides a method of preparing polymer, the method comprising polymerising under the control of a RAFT agent of formula (I) one or more ethylenically unsaturated monomers in a reaction medium selected from an aqueous reaction medium and an organic reaction medium:

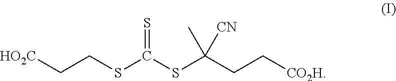 [SMILES: CC(C#N)(CCC(=O)O)SC(=S)SCCC(=O)O]

The present invention further provides polymer of formula (II):

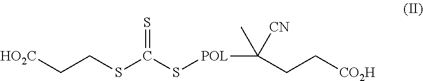

where POL is a polymer chain comprising the polymerised residues of one or more ethylenically unsaturated monomers.

The RAFT agent of formula (I) can advantageously be prepared in high yield and if required subsequently be readily purified to high purity.

The present invention therefore further provides a method of preparing a RAFT agent of formula (I), the method comprising reacting a compound of formula (III) with a compound of formula (IV) in a reaction medium:

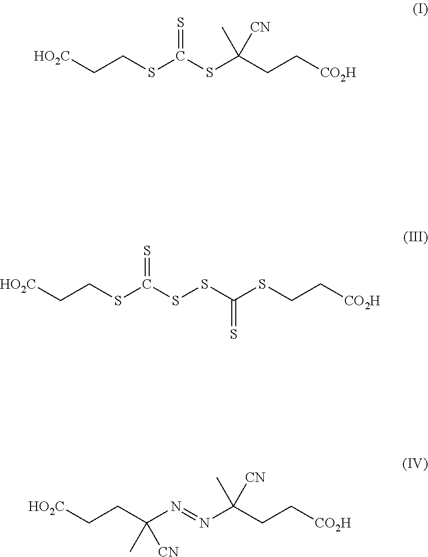 [SMILES: CC(C#N)(CCC(=O)O)SC(=S)SCCC(=O)O ; O=C(O)CCSC(=S)SSC(=S)SCCC(=O)O ; CC(C#N)(CCC(=O)O)/N=N/C(C)(C#N)CCC(=O)O]

Furthermore, the RAFT agent of formula (I) can advantageously be prepared in situ prior to being used for polymerisation without the need to be isolated. In other words, it has been surprisingly found the RAFT agent of formula (I) can be prepared in a reaction medium from precursor compounds, and the so formed agent used in that reaction medium, without being isolated, to form polymer in accordance with the invention. Such a method advantageously provides a simple “one pot” procedure for preparing polymer in accordance with the invention.

The present invention therefore also provides a method of preparing polymer, the method comprising: (a) combining in a reaction medium a compound of formula (III), a compound of formula (IV) and one or more ethylenically unsaturated monomers;

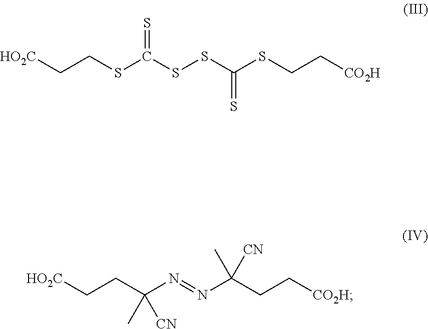 [SMILES: O=C(O)CCSC(=S)SSC(=S)SCCC(=O)O ; CC(C#N)(CCC(=O)O)/N=N/C(C)(C#N)CCC(=O)O]

(b) reacting the compound of formula (III) with the compound of formula (IV) to form a RAFT agent of formula (I); and

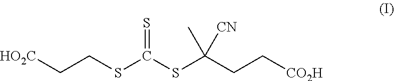 [SMILES: CC(C#N)(CCC(=O)O)SC(=S)SCCC(=O)O]

(c) polymerising in the reaction medium the one or more ethylenically unsaturated monomers under the control of the RAFT agent of formula (I).

The method for preparing the RAFT agent or polymer according to the aforementioned “one pot” procedure can advantageously be performed in a reaction medium selected from an aqueous reaction medium and an organic reaction medium.

The RAFT agent of formula (I) has been found to be particularly well suited for polymerising ethylenically unsaturated monomers commonly referred to in the art as “more activated monomers” (MAM's).

In one embodiment, a method according to the invention comprises polymerising under the control of a RAFT agent of formula (I) one or more ethylenically unsaturated monomers of formula (V):

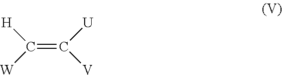

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹, NR^{x}R^{y}, and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

In a further embodiment, in the polymer of formula (II) according to the invention, POL comprises one or more polymerised residues derived from one or more ethylenically unsaturated monomers of formula (V):

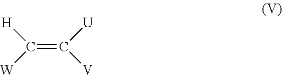

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹, NR^{x}R^{y}, and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

These and other aspects of the invention are described in more detail below.

### DETAILED DESCRIPTION OF THE INVENTION

The present invention provides a RAFT agent of formula (I):

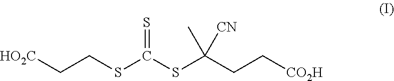 [SMILES: CC(C#N)(CCC(=O)O)SC(=S)SCCC(=O)O]

Those skilled in the art will appreciate the structure of formula (I) may be described as being a trithiocarbonate RAFT agent. By being a “RAFT agent” is meant the agent is capable of participating in a RAFT polymerisation reaction. A RAFT polymerisation reaction is believed to proceed under the control of a RAFT agent according to the mechanism outlined in Scheme 1 (above).

In RAFT polymerisation reactions, one or more ethylenically unsaturated monomers are believed to react under the control of the RAFT agent. By reacting or being polymerised “under the control” of the RAFT agent is meant that reaction of monomer proceeds via a reversible addition-fragmentation chain transfer mechanism.

RAFT agents can advantageously provide excellent control over the reaction process between the agent and monomer. So much so that reaction between a RAFT agent and monomer can provide for a relatively accurate and predetermined number of monomer residue units that become inserted into the RAFT agent. In that context, reference herein to inserted or polymerised residues of monomer is therefore intended to mean residues derived from monomer that participates in a RAFT reaction which become covalently bound to the RAFT agent.

For example, monomer may undergo reaction with a RAFT agent (or fragment thereof) whereby a relatively low number of monomer residue units (e.g. 2-5) are inserted, or a relatively high number of monomer residue units (e.g. 500-1000) are inserted.

While it may be possible to prepare the RAFT agent of formula (I) by a number of synthetic pathways, the method according to the invention has been found to be particularly effective.

According to that method, the RAFT agent of formula (I) is prepared by reacting a compound of formula (III) with a compound of formula (IV) in a reaction medium.

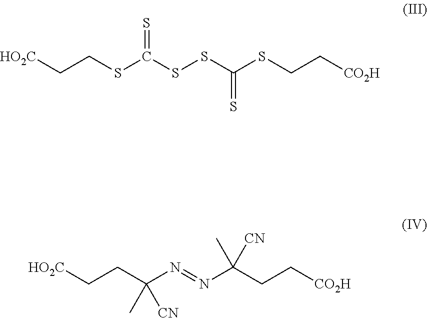 [SMILES: O=C(O)CCSC(=S)SSC(=S)SCCC(=O)O ; CC(C#N)(CCC(=O)O)/N=N/C(C)(C#N)CCC(=O)O]

The compound of formula (III) (i.e. 3,3′-((disulfanne-1,2-dicarbonothioyl)bis(sulfanediyl))dipropionic acid) can itself be readily prepared by reacting 3-mercaptopropanoic acid with carbon disulphide in a suitable reaction medium, for example acetonitrile.

The compound of formula (IV) (i.e. 4,4′-(diazene-1,2-diyl)bis(4-cyanopentanoic acid) is readily available commercially. This compound is also known as 4,4′-azobis(4-cyanovaleric acid).

The RAFT agent of formula (I) is prepared in a reaction medium. The reaction medium can be selected from an aqueous reaction medium and an organic reaction medium. The reaction medium may therefore comprise water, aromatic solvent (e.g. benzene, xylene, toluene), ether solvent (e.g. tetrahydrofuran, diethyl ether, dioxan), ester solvent (e.g. ethyl acetate, butyl acetate) alcohol solvent (e.g. methanol, ethanol, ispropanol), alkylnitrile solvent (e.g. acetonitrile), ketone solvent (e.g. methyl amyl ketone, methyl isobutyl ketone, methyl ethyl ketone, acetone) or combinations thereof.

When preparing the RAFT agent of formula (I), the compound of formula (III) will generally be reacted with the compound of formula (IV) in a mole ratio ranging from about 1:1 to about 1:5, for example from about 1:1 to about 1:2.

To prepare the RAFT agent of formula (I), reaction between compounds of formula (III) and formula (IV) may be promoted by means well known to those skilled in the art. The compound of formula (IV) is a known free radical initiator. The reaction between compounds of formula (III) and formula (IV) will generally be promoted simply by causing the compound of formula (IV) to decompose to produce initiating free radicals. The production of such initiating free radicals may be promoted by any suitable means, for example thermally.

Once prepared, the RAFT agent of formula (I) can be isolated and if required purified for subsequent use. Alternatively, the reaction medium within which the RAFT agent of formula (I) is prepared may be used as a direct source of the RAFT agent (i.e. without the RAFT agent being isolated from the reaction medium).

In contrast with conventional RAFT agents, the RAFT agent of formula (I) not only exhibits suitable reactivity to control the polymerisation of a diverse range of monomers, but the agent surprisingly also exhibits a unique solubility characteristic that enables monomers to be polymerised in either aqueous or organic reaction media.

Those skilled in the art will appreciate that an important criteria for selecting a given conventional RAFT agent is the agent's solubility in the reaction medium within which the polymerisation is to be conducted. In practice, conventional RAFT agents typically only have sufficient solubility in an aqueous or organic reaction media to effectively and efficiently control the polymerisation of monomer according to a RAFT mechanism. Accordingly, different conventional RAFT agents are typically required when performing polymerisations in aqueous and organic reaction media.

The RAFT agent in accordance with the present invention has a unique structure that surprisingly provides it with adequate solubility in both aqueous and organic reaction media to effectively and efficiently control the polymerisation of monomer according to a RAFT mechanism in either medium. The RAFT agent of formula (I) is therefore considerably more versatile than conventional RAFT agents.

Without wishing to be limited by theory, the unique solubility profile of the RAFT agent of formula (I) is believed to stem from the structural features of the agent having a particular balance of both hydrophilic and hydrophobic character. The RAFT agent of formula (I) may therefore be described as having amphiphilic character or properties.

In one embodiment, the reaction medium is an aqueous reaction medium.

In another embodiment, the reaction medium is an organic reaction medium.

Reference herein to the expression “aqueous reaction medium” is intended to mean an aqueous medium within which RAFT polymerisation is performed. Similarly, reference herein to the expression “organic reaction medium” is intended to mean an organic medium within which a RAFT polymerisation is performed. Such reaction media are intended to be liquid reaction media.

By the reaction medium being referred to as an “aqueous” reaction medium is meant that the reaction medium comprises greater than 50 wt %, or at least 60 wt %, or at least 70 wt %, or at least 80 wt %, or at least 90 wt %, or at least 95 wt % water.

By the reaction medium being an “organic” reaction medium is intended to mean the reaction medium comprises greater than 50 wt %, or at least 60 wt %, or at least 70 wt %, or at least 80 wt %, or at least 90 wt %, or at least 95 wt % organic liquid or solvent.

An aqueous reaction medium may comprise a proportion of organic solvent, and an organic reaction medium may comprise a proportion of water. However, those skilled in the art will appreciate that an aqueous reaction medium will have a greater propensity for solubilising more hydrophilic RAFT agents, and an organic reaction medium will have a greater propensity for solubilising more hydrophobic RAFT agents. Having said that, those skilled in the art will also appreciate that an organic reaction medium may present hydrophilic properties (e.g. ethanol) or hydrophobic properties (e.g. toluene). Accordingly, a hydrophilic organic reaction medium will have a greater propensity for solubilising more hydrophilic RAFT agents, and a hydrophobic organic reaction medium will have a greater propensity for solubilising more hydrophobic RAFT agents. The RAFT agent of formula (I) in accordance with the invention has a unique structure that surprisingly affords an appreciable solubility in aqueous and organic (hydrophilic or hydrophobic) reaction media.

In one embodiment, the reaction medium is selected from aqueous, hydrophilic organic and hydrophobic organic reaction media.

Examples of suitable organic liquids or solvents that may be used as an organic reaction medium include, but are not limited to, alcohols, such as methanol, ethanol, 2-propanol and 2-butanol; aromatic hydrocarbons, such as toluene, xylenes or petroleum naphtha; ketones, such as methyl amyl ketone, methyl isobutyl ketone, methyl ethyl ketone or acetone; esters, such as butyl acetate or hexyl acetate; ethers, such as 1,2-dimethoxyethane, tetrahydrofuran and dioxane; glycol ether esters, such as propylene glycol monomethyl ether acetate, alkyl nitriles such as acetonitrile, and ethylenically unsaturated monomer.

An aqueous reaction medium may comprise a hydrophilic organic solvent such as those selected from methanol, ethanol, propanol, acetonitrile, acetone, dioxane, tetrahydrofuran and combinations thereof.

Reference herein to the RAFT agent of formula (I) having sufficient, adequate or appreciable solubility in aqueous and organic reaction media is intended to mean the solubilised concentration of the agent is such that it can effectively and efficiently control the polymerisation of monomer in that media according to a RAFT mechanism.

Generally, the RAFT agent of formula (I) will provide have a solubility of at least 1 mg/L in the reaction medium.

The RAFT agent of formula (I) is used in the method of preparing polymer according to the invention. The method comprises polymerising under the control of the RAFT agent one or more ethylenically unsaturated monomers in a reaction medium selected from an aqueous reaction medium and an organic reaction medium.

Those skilled in the art will appreciate that for the one or more ethylenically unsaturated monomers to undergo RAFT polymerisation they must be of a type that can be polymerised by a free radical process. If desired, the monomers should also be capable of being polymerised with other monomers. The factors which determine copolymerisability of various monomers are well documented in the art. For example, see: Greenlee, R. Z., in Polymer Handbook 3^{rd} Edition (Brandup, J., and Immergut. E. H. Eds) Wiley: New York, 1989 p II/53.

Examples of ethylenically unsaturated monomers include those of formula (VI):

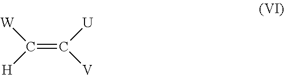

where U and W are independently selected from —CO₂H, —CO₂R¹, —COR¹, —CSR¹, —CSOR¹, —COSR¹, —CONH₂, —CONHR¹, —CONR¹₂, hydrogen, halogen and optionally substituted C₁-C₄ alkyl or U and W form together a lactone, anhydride or imide ring that may itself be optionally substituted, where the optional substituents are independently selected from hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹;
V is selected from hydrogen, R¹, —CO₂H, —CO₂R¹, —COR¹, —CSR¹, —CSOR¹, —COSR¹, —CONH₂, —CONHR¹, —CONR¹₂, —OR¹, —SR¹, —O₂CR¹, —SCOR¹, —NR^{x}R^{y}, and —OCSR¹;
where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and
where R^{x} and R^{y} form together with N an optionally substituted heterocyclic or heteroaryl group.

The or each R¹ in formula (VI) may be independently selected from optionally substituted C₁-C₂₂ alkyl, optionally substituted C₂-C₂₂ alkenyl, optionally substituted C₂-C₂₂ alkynyl, optionally substituted C₆-C₁₈ aryl, optionally substituted C₃-C₁₈ heteroaryl, optionally substituted C₃-C₁₈ carbocyclyl, optionally substituted C₂-C₁₈ heterocyclyl, optionally substituted C₇-C₂₄ arylalkyl, optionally substituted C₄-C₁₈ heteroarylalkyl, optionally substituted C₇-C₂₄ alkylaryl, optionally substituted C₄-C₁₈ alkylheteroaryl, and an optionally polymer chain.

The or each R¹ in formula (VI) may also be independently selected from optionally substituted C₁-C₆ alkyl.

Examples of optional substituents for R¹ in formula (VI) include those selected from epoxy, hydroxy, alkoxy, acyl, acyloxy, formyl, alkylcarbonyl, carboxy, sulfonic acid, alkoxy- or aryloxy-carbonyl, isocyanato, cyano, silyl, halo, amino, including salts and derivatives thereof. Examples polymer chains include those selected from polyalkylene oxide, polyarylene ether and polyalkylene ether.

Specific examples of monomers of formula (VI) include maleic anhydride, N-alkylmaleimide, N-arylmaleimide, dialkyl fumarate and cyclopolymerisable monomers, acrylate and methacrylate esters, acrylic and methacrylic acid, styrene, acrylamide, methacrylamide, and methacrylonitrile, mixtures of these monomers, and mixtures of these monomers with other monomers.

Other specific examples of monomers of formula (VI) include: methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, N-vinylcarbazole, butadiene, ethylene and chloroprene. This list is not exhaustive.

The R and Z groups of conventional RAFT agents (see Schemel) for use in a given polymerisation reaction are typically selected having regard to the type of monomers that are to be polymerised. For example, it is known in the art that Z groups that afford dithiocarbamate and xanthate RAFT agents can in general be used for controlling the polymerisation of monomers that produce relatively unstabilised propagating radicals (i.e. less activated monomers (LAM's) such as vinyl esters and vinyl amides), whereas Z groups that form dithioester and trithiocarbonate RAFT agents can in general be used for controlling the polymerisation of monomers that produce relatively stabilised propagating radicals (i.e. more activated monomers (MAM's) such as styrenes, acrylates, acrylamides, methacrylates and methacrylamides). Consequently, most conventional RAFT agents will generally be unsuitable for use in controlling the polymerisation of both less activated and more activated monomers (i.e. monomers having markedly disparate reactivities e.g. styrene and vinyl acetate).

In one embodiment, a method according to the invention comprises polymerising under the control of a RAFT agent of formula (I) one or more ethylenically unsaturated monomers of formula (V):

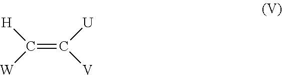

where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹, NR^{x}R^{y}, and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

Those skilled in the art will appreciate there can also exist a further disparate reactivity of monomers even within the class of MAM's. For example, MAM's that give rise to a secondary incipient radical (e.g. acrylates) can exhibit disparate reactivity compared to MAM's that give rise to a tertiary incipient radical (e.g. methacrylates). A given conventional RAFT agent may therefore be ineffective at polymerising monomers that give rise to a secondary incipient radical (e.g. acrylates) and also monomers that give rise to a tertiary incipient radical (e.g. methacrylates).

The RAFT agent of formula (I) in accordance with the invention has been found to be well suited at polymerising MAM's, and most notably MAM's that give rise to a secondary incipient radical (e.g. acrylates) and also MAM's that give rise to a tertiary incipient radical (e.g. methacrylates).

By an ethylenically unsaturated monomer providing for a “secondary incipient radical” or a “tertiary incipient radical” is meant that a secondary or tertiary radical, respectively, is produced by the monomer upon the ethylenically unsaturated functional group undergoing a free radical addition reaction.

In one embodiment, an ethylenically unsaturated monomer used in accordance with the invention is selected to provide for a secondary incipient radical.

In another embodiment, an ethylenically unsaturated monomer used in accordance with the invention is selected to provide for a tertiary incipient radical.

Examples of ethylenically unsaturated monomers that provide for a secondary incipient radical include those of formula (Va):

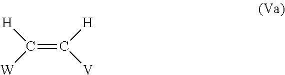

where W is H or forms together with V a lactone, anhydride or imide ring; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), NR^{x}R^{y}, SOR¹ and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

Examples of ethylenically unsaturated monomers that provide for a tertiary incipient radical include those of formula (Vb):

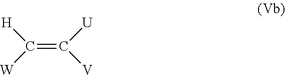

where W is H or forms together with V a lactone, anhydride or imide ring; U is selected from C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

Examples of monomers of formula (Va) include acrylates, styrenics, acrylic acid, vinyl aromatics and heteroaromatics, conjugated dienes, acrylamides, acrylonitrile, maleic anhydride and maleimides, vinyl sulphones, vinyl sulphoxides, n-vinyl carbazole, vinyl phosphinates, and vinyl phosphonates.

Examples of monomers of formula (Vb) include methacrylates, methacrylic acid, methacrylamides, and alpha-methyl styrenics.

Specific examples of monomers of formula (Va) include methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, N-methylolacrylamide, N-ethylolacrylamide, vinyl benzoic acid (all isomers), diethylamino styrene (all isomers), p-vinylbenzene sulfonic acid, p-vinylbenzene sulfonic sodium salt, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, butadiene, chloroprene, acenapthalene, vinylnapthalene, vinylbiphenyl, vinyl azlactone; 1-vinylimidazole; 2-vinylpyridine, 4-vinyl pyridine, n-vinyl carbazole and vinylferrocene.

Specific examples of monomers of formula (Vb) include methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, methacrylamide, N-tert-butylmethacrylamide, N-n-butylmethacrylamide, N-methylolmethacrylamide, N-ethylolmethacrylamide, alpha-methylvinyl benzoic acid (all isomers), diethylamino alpha-methylstyrene (all isomers), trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, and diisopropoxysilylpropyl methacrylate.

A further summary of monomers amenable to RAFT polymerisation can also be found in reviews such as Moad et al, Polymer 49 (2008), 1079-1131.

A particular advantage afforded by RAFT polymerisation is the ability to produce polymer having a well defined molecular architecture, a predetermined molecular weight and a narrow molecular weight distribution or low dispersity (Ð).

The RAFT agent in accordance with the invention can advantageously provide for polymer having a low dispersity of (Ð).

In one embodiment, polymer produced in accordance with the method of the invention, or polymer according to the invention, has a dispersity (Ð) of less than 1.7, or less than 1.6, or less than 1.5, or less than 1.4, or less than 1.3, or less than 1.2, or less than 1.1.

Polymer produced in accordance with the method of the invention, or polymer according to the invention, can advantageously have a dispersity (Ð) of less than 1.4, or less than 1.3, or less than 1.2, or less than 1.1.

As used herein, the dispersity (Ð) of polymer is determined according to equation (1):

Ð=M_{w}/Mₙ (I)
where M_{w} is the mass average molecular weight, and Mₙ is the number average molecular weight.

M_{w} and Mₙ, referred to herein are intended to be that determined by Size Exclusion Chromatography (SEC) using poly(methyl methacrylate) standards.

The method of preparing polymer in accordance with the invention can advantageously be performed using techniques and reagents well known to those skilled in the art.

Polymerisation of the monomers will usually require initiation from a source of free radicals. The source of initiating radicals can be provided by any suitable method of generating free radicals, such as the thermally induced homolytic scission of suitable compound(s) (e.g. thermal initiators such as peroxides, peroxyesters, or azo compounds), the spontaneous generation from monomers (e.g. styrenics), redox initiating systems, photochemical initiating systems or high energy radiation such as electron beam, X- or gamma-radiation. The initiating system is chosen such that under the reaction conditions there is no substantial adverse interaction of the initiator or the initiating radicals with the RAFT agent under the conditions of the reaction. The initiator ideally should also have the requisite solubility in the reaction medium.

Initiation of polymerisation according to a method of the invention may simply be promoted thermally and/or photolytically.

In one embodiment, polymerisation according to a method of the invention is promoted thermally and/or photolytically.

Polymerisation according to a method of the invention can advantageously be promoted via free radicals generated formed during preparation of the RAFT agent of formula (I) (see below for more detail).

Thermal initiators are chosen to have an appropriate half life at the temperature of polymerisation. These initiators can include one or more of the following compounds:
2,2′-azobis(isobutyronitrile), 2,2′-azobis(2-cyanobutane), dimethyl 2,2′-azobis(isobutyrate), 4,4′-azobis(4-cyanovaleric acid), 1,1′-azobis(cyclohexanecarbonitrile), 2-(t-butylazo)-2-cyanopropane, 2,2′-azobis{2-methyl-N-[1,1-bis(hydroxymethyl)-2-hydroxyethyl]propionamide}, 2,2′-azobis[2-methyl-N-(2-hydroxyethyl)propionamide], 2,2′-azobis(N,N′-dimethyleneisobutyramidine) dihydrochloride, 2,2′-azobis(2-amidinopropane) dihydrochloride, 2,2′-azobis(N,N′-dimethyleneisobutyramidine), 2,2′-azobis{2-methyl-N-[1,1-bis(hydroxymethyl)-2-hydroxyethyl]propionamide}, 2,2′-azobis{2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, 2,2′-azobis(2,2,4-trimethylpentane), 2,2′-azobis(2,4-dimethyl-4-methoxyvaleronitrile), 2,2′-azobis(2-methylpropane), t-butyl peroxyacetate, t-butyl peroxybenzoate, t-butyl peroxyneodecanoate, t-butylperoxy isobutyrate, t-amyl peroxypivalate, t-butyl peroxypivalate, diisopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl hyponitrite, dicumyl hyponitrite. This list is not exhaustive.

Photochemical initiator systems are chosen to have the requisite solubility in the reaction medium and have an appropriate quantum yield for radical production under the conditions of the polymerisation. Examples include benzoin derivatives, benzophenone, acyl phosphine oxides, and photo-redox systems.

Redox initiator systems are chosen to have the requisite solubility in the reaction medium and have an appropriate rate of radical production under the conditions of the polymerisation; these initiating systems can include, but are not limited to, combinations of the following oxidants and reductants:
oxidants: potassium, peroxydisulfate, hydrogen peroxide, t-butyl hydroperoxide.
reductants: iron (II), titanium (III), potassium thiosulfite, potassium bisulfite.

Other suitable initiating systems are described in recent texts. See, for example, Moad and Solomon “the Chemistry of Free Radical Polymerisation”, Pergamon, London, 1995, pp 53-95.

Reaction conditions for the polymerisation should be chosen such that the ratio of the total number of initiator-derived radicals to the number of RAFT agent molecules is maintained at a minimum value consistent with achieving an acceptable rate of polymerisation. Generally, such a ratio is less than 1:1, or less than 1:10, or in the range of 1:10 to 1:5000.

With the above consideration in mind, the initiator concentration will be chosen so as to give an acceptable rate of polymerization of the specific monomer or monomer combination.

Those skilled in the art will appreciate that in the application of RAFT agents the chain transfer constant is considered an important parameter of the addition-fragmentation steps that occur in the polymerisation process. A consideration of chain transfer constants for RAFT agents is given in WO 98/01478.

The method of the invention may be carried out using solution, emulsion, bulk or suspension polymerisation techniques in either batch, semi-batch, continuous, or feed modes.

Where polymerisation is performed as a bulk polymerisation, it will be appreciated the reaction medium will be monomer per se which in effect functions as an organic reaction medium.

As it will be appreciated by those skilled in the art, the choice of polymerisation conditions can be important when performing RAFT polymerisation. The reaction temperature may influence the rate parameters discussed above. For example, higher reaction temperatures can increase the rate of fragmentation. Conditions should be chosen such that the number of polymer chains formed from initiator-derived radicals is minimised to an extent consistent with obtaining an acceptable rate of polymerisation. Termination of polymerisation by radical-radical reaction will lead to chains which contain no active group and therefore cannot be reactivated. The rate of radical-radical termination is proportional to the square of the radical concentration. These reaction conditions may therefore require careful choice of the initiator concentration and, where appropriate the rate of the initiator feed.

If present, it may also desirable to choose other components of the reaction medium (for example, solvent, surfactant, additive, and initiator) such that they have a low transfer constant towards the propagating radical. Chain transfer to these species will lead to the formation of polymer chains which do not contain the active RAFT group.

As a general guide in choosing conditions for the synthesis of narrow polydispersity polymers, the concentration of initiator(s) and other reaction conditions (solvent(s) if any, reaction temperature, reaction pressure, surfactants if any, other additives) should be chosen such that the molecular weight of polymer formed in the absence of the RAFT agent is at least twice that formed in its presence. In polymerisations where termination is solely by disproportionation, this equates to choosing an initiator concentration such that the total moles of initiating radicals formed during the polymerisation is less than 0.5 times that of the total moles of RAFT agent. It can be desirable to choose conditions such that the molecular weight of polymer formed in the absence of the RAFT agent is at least 5-fold that formed in its presence ([initiating radicals]/[RAFT agent]<0.2).

Thus, the dispersity (Ð) can be controlled by varying the number of moles of RAFT agent to the number of moles initiating radicals. Lower dispersities (Ð) can be obtained by increasing this ratio; higher dispersities (Ð) can be obtained by decreasing this ratio.

Polymerisation will generally be carried out at temperatures in the range of −20 to 200° C., for example in the range of 40 to 160° C. The polymerisation temperature may be chosen taking into consideration the specific monomer(s) being polymerised and other components of the polymerisation or reaction medium.

In the case of emulsion or suspension polymerisation the reaction medium will often be predominantly water and conventional stabilisers, dispersants and other additives may also be present.

For solution polymerisation, the reaction medium can be chosen from a wide range of media to suit the monomer(s) being used. For example, water; alcohols, such as methanol, ethanol, 2-propanol and 2-butanol; aromatic hydrocarbons, such as toluene, xylenes or petroleum naphtha; ketones, such as methyl amyl ketone, methyl isobutyl ketone, methyl ethyl ketone or acetone; esters, such as butyl acetate or hexyl acetate; ethers, such as 1,2-dimethoxyethane, tetrahydrofuran and dioxane; and glycol ether esters, such as propylene glycol monomethyl ether acetate.

The RAFT agent of formula (I) can advantageously be prepared without being isolated and used directly for preparing polymer according to the invention.

Accordingly, a method of preparing polymer according to the invention may further comprise preparing the RAFT agent of formula (I) by reacting a compound of formula (III) with a compound of formula (IV) in a reaction medium;

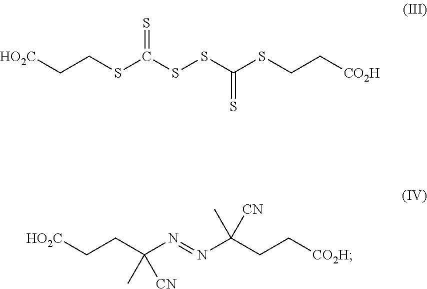 [SMILES: O=C(O)CCSC(=S)SSC(=S)SCCC(=O)O ; CC(C#N)(CCC(=O)O)/N=N/C(C)(C#N)CCC(=O)O]

combining the one or more ethylenically unsaturated monomers with the reaction medium comprising the so formed RAFT agent of formula (I), and polymerising in the reaction medium the one or more ethylenically unsaturated monomers under the control of the RAFT agent of formula (I).

The one or more ethylenically unsaturated monomers may be combined with the reaction medium comprising RAFT agent of formula (I) by any suitable means. For example, the monomer may be introduced to the reaction medium comprising RAFT agent of formula (I), or the reaction medium comprising RAFT agent of formula (I) may be introduced to the monomer. Techniques and equipment well known to those skilled in the art can be used for combining one or more ethylenically unsaturated monomers with the reaction medium comprising RAFT agent of formula (I).

The one or more ethylenically unsaturated monomers to be polymerised can advantageously also be present in the reaction medium at the time of preparing the RAFT agent of formula (I). In that case, polymer may be produced in accordance with the invention in a simple and streamline “one pot” procedure.

The present invention therefore also provides a method of preparing polymer, the method comprising: (a) combining in a reaction medium a compound of formula (III), a compound of formula (IV) and one or more ethylenically unsaturated monomers;

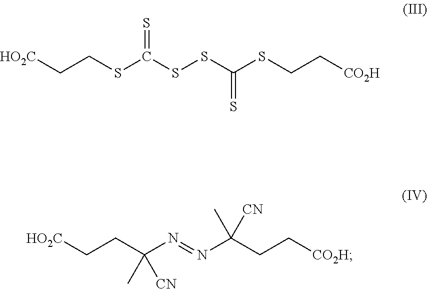 [SMILES: O=C(O)CCSC(=S)SSC(=S)SCCC(=O)O ; CC(C#N)(CCC(=O)O)/N=N/C(C)(C#N)CCC(=O)O]

(b) reacting the compound of formula (III) with the compound of formula (IV) to form a RAFT agent of formula (I); and

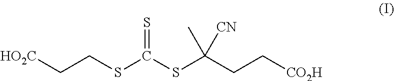 [SMILES: CC(C#N)(CCC(=O)O)SC(=S)SCCC(=O)O]

(c) polymerising in the reaction medium the one or more ethylenically unsaturated monomers under the control of the RAFT agent of formula (I).

This “one pot” procedure can advantageously be performed in a reaction medium selected from an aqueous reaction medium and an organic reaction medium.

Where the one or more ethylenically unsaturated monomers are present in the reaction medium at the time when the compound of formula (III) is reacted with the compound of formula (IV) to form the RAFT agent of formula (I), free radicals produced as part of the formation of the RAFT agent of formula (I) can advantageously also promote polymerisation of the monomers under the control of the so formed RAFT agent. Preparing polymer according to that method can therefore proceed simply by, for example, heating the reaction medium comprising the compound of formula (III), the compound of formula (IV) and the one or more ethylenic ally unsaturated monomers.

Polymer according to the invention, or that produced in accordance with the method of the invention, comprises the reaction residue of a RAFT agent of formula (I), together with a polymer chain comprising the polymerised residues of one or more ethylenically unsaturated monomers as herein described. The so formed polymer may be in the form of a homopolymer, copolymer, block copolymer, multi block copolymer, gradient copolymer or random or statistical copolymer.

As used herein, the term “alkyl”, used either alone or in compound words denotes straight chain, branched or cyclic alkyl, preferably C₁₋₂₀ alkyl, e.g. C₁₋₁₀ or C₁₋₆ Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo). Preferred halogens are chlorine, bromine or iodine.

The term “aryl” (or “carboaryl)” denotes any of single, polynuclear, conjugated and fused residues of aromatic hydrocarbon ring systems (e.g C₆₋₁₈ aryl). Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl. A carbocyclyl group may be optionally substituted by one or more optional substituents as herein defined. The term “carbocyclylene” is intended to denote the divalent form of carbocyclyl.

The term “heterocyclyl” includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, carbazole, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl. A heteroaryl group may be optionally substituted by one or more optional substituents as herein defined. The term “heteroarylene” is intended to denote the divalent form of heteroaryl.

The term “acyl” either alone or in compound words denotes a group containing the moiety C═O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{x} residue may be optionally substituted as described herein.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl, aryl, heteroaryl, heterocyclyl, carbocyclyl and aralkyl. Examples of preferred R^{f} include C₁₋₂₀alkyl, phenyl and benzyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In a preferred embodiment at least one R^{f} is hydrogen. In another form, both R^{f} are hydrogen.

The term, “amino” is used here in its broadest sense as understood in the art and includes groups of the formula NR^{a}R^{b} wherein R^{a} and R^{b} may be independently selected from hydrogen, alkyl, alkenyl, alkynyl, aryl, carbocyclyl, heteroaryl, heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(O)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{a}R^{b}, wherein R^{a} and R^{b} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g. CO₂phenyl), CO₂aralkyl (e.g. CO₂ benzyl).

In this specification “optionally substituted” is taken to mean that a group may or may not be substituted or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate and phosphate groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

Preferred optional substituents include alkyl, (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), nitro, formyl, —C(O)-alkyl (e.g. C₁₋₆ alkyl, such as acetyl), O—C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g. C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl) aminoalkyl (e.g., HN C₁₋₆ alkyl-, C₁₋₆alkylHN—C₁₋₆ alkyl- and (C₁₋₆ alkyl)₂N—C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R(O)SC₁₋₆ alkyl, such as C₁₋₆ alkyl(O)SC₁₋₆ alkyl-), sulfonylalkyl (e.g., R(O)₂SC₁₋₆ alkyl-such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-).

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

For monovalent substituents, terms written as “[group A][group B]” refer to group A when linked by a divalent form of group B. For example, “[group A][alkyl]” refers to a particular group A (such as hydroxy, amino, etc.) when linked by divalent alkyl, i.e. alkylene (e.g. hydroxyethyl is intended to denote HO—CH₂—CH—). Thus, terms written as “[group]oxy” refer to a particular group when linked by oxygen, for example, the terms “alkoxy” or “alkyloxy”, “alkenoxy” or “alkenyloxy”, “alkynoxy” or alkynyloxy“, “aryloxy” and “acyloxy”, respectively, denote alkyl, alkenyl, alkynyl, aryl and acyl groups as hereinbefore defined when linked by oxygen. Similarly, terms written as “[group]thio” refer to a particular group when linked by sulfur, for example, the terms “alkylthio”, “alkenylthio”, alkynylthio” and “arylthio”, respectively, denote alkyl, alkenyl, alkynyl and aryl groups as hereinbefore defined when linked by sulfur.

The invention will hereinafter be described with reference to the following non-limiting examples.

### EXAMPLES

### Preparative Example 1

### Preparation of 3,3′-((disulfanne-1,2-dicarbonothioyl)bis(sulfanediyl))dipropionic Acid

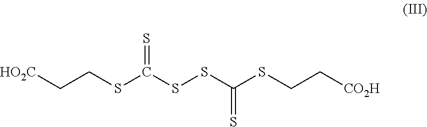 [SMILES: O=C(O)CCSC(=S)SSC(=S)SCCC(=O)O]

A mixture of 3-mercaptopropanoic acid (10.6 g) and potassium carbonate (14.1 g) in acetonitrile (800 mL) was stirred at room temperature for 2 hours, and then treated with carbon disulfide (9 mL) and stirred at room temperature for 18 hours. The reaction mixture was then filtered and the solids collected washed with water and dried. 24.4 g of yellow solid was recovered. This material was then dissolved in water (250 mL) and treated with iodine (12.7 g), in portions, whilst being stirred, over 1.5 hours. When the iodine had reacted, the reaction mixture was treated with conc HCl (12 mL), then extracted with ethyl acetate (200 mL). This latter phase was then washed with brine (30 mL), dried (MgSO4), and then evaporated to dryness, leaving behind 13.7 g of a yellow solid. This material was used further without additional treatment.

### Example 2

### Preparation of 4-((((2-carboxyethyl)thio)carbonothioyl)thio)-4-cyanopentanoic Acid

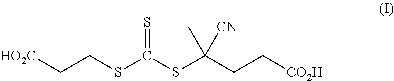 [SMILES: CC(C#N)(CCC(=O)O)SC(=S)SCCC(=O)O]

A mixture of crude 3,3′-((disulfanne-1,2-dicarbonothioyl)bis(sulfanediyl))dipropionic acid (1 g) form Example 1 and 4,4′-(diazene-1,2-diyl)bis(4-cyanopentanoic acid) (1.16 g) in acetonitrile (12 mL) was heated under reflux, with stirring, for 18 hours, then cooled and the solvent removed under reduced pressure. The remaining oil was purified by radial chromatography using methanol in dichloromethane mixtures.

¹H NMR (400 MHz, D₆-Acetone): 1.91 (s, 3H, CH₃), 2.38-2.62 (m, 4H, 2×CH₂) 2.79 (t, 2H, CH₂), 3.62 (t, 2H, CH₂). ¹³C NMR (100 MHz, D₆-Acetone): 24.0, 29.01, 31.7, 32.0, 33.7, 47.1, 118.9, 171.9, 172.1, 218.5. Log P=1.90 (obtained from ChemDraw).

### Polymer Preparation

In all instances, monomers were purified (to remove inhibitors) immediately prior to use. Polymerizations were performed in vials, degassing was accomplished by nitrogen purging over an extended period (typically 20 minutes). Once degassing was complete, the ampoules were sealed under nitrogen and completely submerged in an oil bath or exposed to microwave radiation at the specified temperature for the specified times. The percentage conversions were calculated spectroscopically or gravimetrically unless otherwise indicated.

### Example 3

### Preparation of poly(styrene-alt-maleic anhydride) Using 4-((((2-carboxyethyl)thio)carbonothioyl)thio)-4-cyanopentanoic Acid (1) at 80° C. in Ethyl Acetate

A solution containing styrene (1.031 mL), 4,4′-azobis(4-cyanovaleric acid (5.2 mg), 4-((((2-carboxyethyl)thio)carbonothioyl)thio)-4-cyanopentanoic acid (I) (99.4 mg), and ethyl acetate (3.373 mL) was prepared. All components dissolved completely to give a clear yellow solution. The resulting mixture was degassed, sealed and heated at 80° C. in a microwave reactor for 90 minutes. The volatiles were removed in vacuo to give poly(styrene-alt-maleic anhydride) at 87% conversion (based on the consumption of styrene and maleic anhydride), with Mₙ 9,726, M_{w}/Mₙ 1.11.

### Example 4

### Preparation of poly(styrene-alt-maleic anhydride) Using 4-((((2-carboxyethyl)thio)carbonothioyl)thio)-4-cyanopentanoic Acid (1) at 80° C. in Ethyl Acetate

A solution containing styrene (1.031 mL), maleic anhydride (0.883 g), 4,4′-azobis(4-cyanovaleric acid (5.2 mg), 4-((((2-carboxyethyl)thio)carbonothioyl)thio)-4-cyanopentanoic acid (I) (11.1 mg), and ethyl acetate (3.373 mL) was prepared. All components dissolved completely to give a clear yellow solution. The resulting mixture was degassed, sealed and heated at 80° C. in a microwave reactor for 90 minutes. The volatiles were removed in vacuo to give poly(styrene-alt-maleic anhydride) at 99% conversion (based on the consumption of styrene and maleic anhydride), with Mₙ 67,491, M_{w}/Mₙ 1.19.

### Example 5

### Preparation of poly(N,N-dimethylacrylamide) Using 4-((((2-carboxyethyl)thio)carbonothioyl)thio)-4-cyanopentanoic Acid (1) at 80° C. in Water

A solution containing N,N-dimethylacrylamide (11.542 mL), 4,4′-azobis(4-cyanovaleric acid (94.5 mg), 4-((((2-carboxyethyl)thio)carbonothioyl)thio)-4-cyanopentanoic acid (I) (689.7 mg), and water (28.459 mL) was prepared. All components dissolved completely to give a clear yellow solution. The resulting mixture was degassed, sealed and heated at 80° C. in a microwave reactor for 90 minutes. The volatiles were removed in vacuo to give poly(N,N-dimethylacrylamide) at 99% conversion (based on the consumption N,N-dimethylacrylamide), with Mₙ 6,454, M_{w}/Mₙ 1.10.

### Example 6

### Preparation of poly(N,N-dimethylacrylamide) Using 4-((((2-carboxyethyl)thio)carbonothioyl)thio)-4-cyanopentanoic Acid (1) at 100° C. in Acetonitrile

A solution containing N,N-dimethylacrylamide (0.618 mL), 1,1′-azobis(cyclohexanecarbonitrile) (2.93 mg), 4-((((2-carboxyethyl)thio)carbonothioyl)thio)-4-cyanopentanoic acid (I) (36.89 mg), and acetonitrile (1.382 mL) was prepared. All components dissolved completely to give a clear yellow solution. The resulting mixture was degassed, sealed and heated at 100° C. in a microwave reactor for 60 minutes. The volatiles were removed in vacuo to give poly(N,N-dimethylacrylamide) at >99% conversion (based on the consumption of N,N-dimethylacrylamide), with Mₙ 5,436, M_{w}/Mₙ 1.13.

### Example 7

### Preparation of poly(N,N-dimethylacrylamide) Using 4-((((2-carboxyethyl)thio)carbonothioyl)thio)-4-cyanopentanoic Acid (1) at 100° C. in Acetonitrile

A solution containing N,N-dimethylacrylamide (0.618 mL), 1,1′-azobis(cyclohexanecarbonitrile) (1.47 mg), 4-((((2-carboxyethyl)thio)carbonothioyl)thio)-4-cyanopentanoic acid (I) (18.44 mg), and acetonitrile (1.382 mL) was prepared. All components dissolved completely to give a clear yellow solution. The resulting mixture was degassed, sealed and heated at 100° C. in a microwave reactor for 60 minutes. The volatiles were removed in vacuo to give poly(N,N-dimethylacrylamide) at 99% conversion (based on the consumption of N,N-dimethylacrylamide), with Mₙ 11,186, M_{w}/Mₙ 1.12.

### Example 8

### Preparation of poly(methylacrylate) Using 4-((((2-carboxyethyl)thio)carbonothioyl)thio)-4-cyanopentanoic Acid (1) at 100° C. in Acetonitrile

A solution containing methylacrylate (0.540 mL), 1,1′-azobis(cyclohexanecarbonitrile) (1.27 mg), 4-((((2-carboxyethyl)thio)carbonothioyl)thio)-4-cyanopentanoic acid (I) (16.4 mg), and acetonitrile (1.460 mL) was prepared. All components dissolved completely to give a clear yellow solution. The resulting mixture was degassed, sealed and heated at 100° C. in a microwave reactor for 60 minutes. The volatiles were removed in vacuo to give poly(methylacrylate) at 76% conversion (based on the consumption of methylacrylate), with Mₙ 8,532, M_{w}/Mₙ 1.15.

### Example 9

### Preparation of poly(methylmethacrylate) Using 4-((((2-carboxyethyl)thio)carbonothioyl)thio)-4-cyanopentanoic Acid (1) at 100° C. in Acetonitrile

A solution containing methylmethacrylate (0.642 mL), 1,1′-azobis(cyclohexanecarbonitrile) (1.47 mg), 4-((((2-carboxyethyl)thio)carbonothioyl)thio)-4-cyanopentanoic acid (I) (18.44 mg), and acetonitrile (1.358 mL) was prepared. All components dissolved completely to give a clear yellow solution. The resulting mixture was degassed, sealed and heated at 100° C. in a microwave reactor for 6 hrs. The volatiles were removed in vacuo to give poly(methylmethacrylate) at 76% conversion (based on the consumption of methylmethacrylate), with Mₙ 8,012, M_{w}/Mₙ 1.13.

### Example 10

### Preparation of poly(methylmethacrylate) Using 4-((((2-carboxyethyl)thio)carbonothioyl)thio)-4-cyanopentanoic Acid (1) at 100° C. in Ethyl Acetate

A solution containing methylmethacrylate (0.749 mL), 1,1′-azobis(cyclohexanecarbonitrile) (2.57 mg), 4-((((2-carboxyethyl)thio)carbonothioyl)thio)-4-cyanopentanoic acid (I) (16.14 mg), and ethyl acetate (1.251 mL) was prepared. All components dissolved completely to give a clear yellow solution. The resulting mixture was degassed, sealed and heated at 100° C. in a microwave reactor for 4 hrs. The volatiles were removed in vacuo to give poly(methylmethacrylate) at 72% conversion (based on the consumption of methylmethacrylate), with Mₙ 11,133, M_{w}/Mₙ 1.14.

### Example 11

### Preparation of poly(methacrylic acid) Using 4-((((2-carboxyethyl)thio)carbonothioyl)thio)-4-cyanopentanoic Acid (1) at 80° C. in Water

A solution containing methacrylic acid (0.509 mL), 4,4′-azobis(4-cyanovaleric acid (3.60 mg), 4-((((2-carboxyethyl)thio)carbonothioyl)thio)-4-cyanopentanoic acid (I) (32.94 mg), and water (1.491 mL) was prepared. All components dissolved completely to give a clear yellow solution. The resulting mixture was degassed, sealed and heated at 80° C. in a microwave reactor for 60 minutes. The volatiles were removed in vacuo to give poly(methacrylic acid) at 97% conversion (based on the consumption of methacrylic acid), with Mₙ 7,549, M_{w}/Mₙ 1.06.

### Example 12

### Preparation of poly(N,N-dimethylacrylamide-co-hydroxyethylacrylamide) Using 4-((((2-carboxyethyl)thio)carbonothioyl)thio)-4-cyanopentanoic Acid (1) at 80° C. in Water

A solution containing N,N-dimethylacrylamide (0.361 mL), hydroxyethylacrylamide (0.363 mL), 4,4′-azobis(4-cyanovaleric acid) (1.96 mg), 4-((((2-carboxyethyl)thio)carbonothioyl)thio)-4-cyanopentanoic acid (I) (21.52 mg), and water (1.277 mL) was prepared. All components dissolved completely to give a clear yellow solution. The resulting mixture was degassed, sealed and heated at 80° C. in a microwave reactor for 90 minutes. The volatiles were removed in vacuo to give poly(N,N-dimethylacrylamide-co-hydroxyethylacrylamide) at 99% conversion (based on the consumption of 99% of N,N-dimethylacrylamide and 99% of hydroxyethylacryamide), with Mₙ 11,946, M_{w}/Mₙ 1.09.

### Example 13

### Preparation of poly(N,N-dimethylacrylamide-co-acrylic acid) Using 4-((((2-carboxyethyl)thio)carbonothioyl)thio)-4-cyanopentanoic Acid (1) at 80° C. in Water

A solution containing N,N-dimethylacrylamide (0.927 mL), acrylic acid (0.627 mL), 4,4′-azobis(4-cyanovaleric acid (5.4 mg), 4-((((2-carboxyethyl)thio)carbonothioyl)thio)-4-cyanopentanoic acid (I) (31.3 mg), and water (3.456 mL) was prepared. All components dissolved completely to give a clear yellow solution. The resulting mixture was degassed, sealed and heated at 80° C. in a microwave reactor for 30 minutes. The volatiles were removed in vacuo to give poly(N,N-dimethylacrylamide-co-acrylic acid) at 96% conversion (based on the consumption of 95% of N,N-dimethylacrylamide and 96% of acrylic acid), with Mₙ 50,996, M_{w}/Mₙ 1.19.

### Example 14

### Preparation of poly(acrylic acid-co-methacrylic acid) Using 4-((((2-carboxyethyl)thio)carbonothioyl)thio)-4-cyanopentanoic Acid (1) at 85° C. in Water

A solution containing acrylic acid (0.617 mL), methacrylic acid (0.763 mL), 4,4′-azobis(4-cyanovaleric acid) (5.2 mg), 4-((((2-carboxyethyl)thio)carbonothioyl)thio)-4-cyanopentanoic acid (I) (29.9 mg), and water (3.620 mL) was prepared. All components dissolved completely to give a clear yellow solution. The resulting mixture was degassed, sealed and heated at 85° C. in a microwave reactor for 60 minutes. The volatiles were removed in vacuo to give poly(acrylic acid-co-methacrylic acid) at 95% conversion (based on the consumption of 90% of acrylic acid and 99% of methacrylic acid), with Mₙ 23,499, M_{w}/Mₙ 1.26.

### Example 15

### Preparation of poly(N,N-dimethylacrylamide) Using 3,3′-((disulfanne-1,2-dicarbonothioyl)bis(sulfanediyl))dipropionic Acid (III) at 100° C. in Acetone

A solution containing N,N-dimethylacrylamide (0.595 mL), 4,4′-azobis(4-cyanovaleric acid) (3.36 mg), 3,3′-((disulfanne-1,2-dicarbonothioyl)bis(sulfanediyl))dipropionic acid (III) (21.75 mg), and acetone (1.092 mL) was prepared. All components dissolved completely to give a clear yellow solution. The resulting mixture was degassed, sealed and heated at 100° C. in a microwave reactor for 60 minutes. The volatiles were removed in vacuo to give poly(N,N-dimethylacrylamide) at >99% conversion (based on the consumption of N,N-dimethylacrylamide), with Mₙ 12,464, M_{w}/Mₙ 1.74.

### Example 16

### Preparation of poly(N,N-dimethylacrylamide) Using 3,3′-((disulfanne-1,2-dicarbonothioyl)bis(sulfanediyl))dipropionic Acid (III) at 100° C. in Acetone

A solution containing N,N-dimethylacrylamide (0.595 mL), 4,4′-azobis(4-cyanovaleric acid) (16.82 mg), 3,3′-((disulfanne-1,2-dicarbonothioyl)bis(sulfanediyl))dipropionic acid (III) (21.75 mg), and acetone (1.092 mL) was prepared. All components dissolved completely to give a clear yellow solution. The resulting mixture was degassed, sealed and heated at 100° C. in a microwave reactor for 60 minutes. The volatiles were removed in vacuo to give poly(N,N-dimethylacrylamide) at >99% conversion (based on the consumption of N,N-dimethylacrylamide), with Mₙ 9,984, M_{w}/Mₙ 1.56.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A RAFT agent of formula (I):

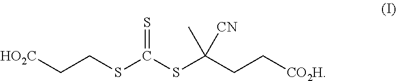 [SMILES: CC(C#N)(CCC(=O)O)SC(=S)SCCC(=O)O]

2. A method of preparing a RAFT agent of formula (I), the method comprising reacting a compound of formula (III) with a compound of formula (IV) in a reaction medium:

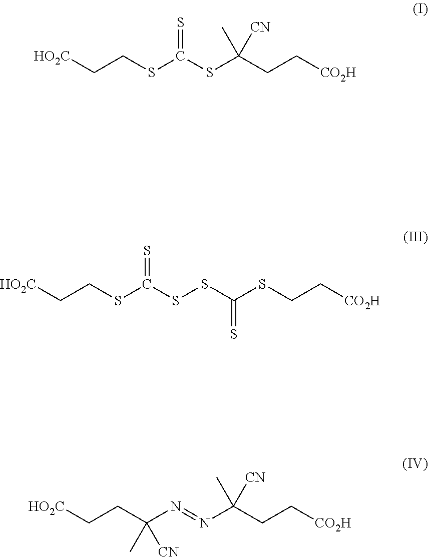 [SMILES: CC(C#N)(CCC(=O)O)SC(=S)SCCC(=O)O ; O=C(O)CCSC(=S)SSC(=S)SCCC(=O)O ; CC(C#N)(CCC(=O)O)/N=N/C(C)(C#N)CCC(=O)O]

3. The method according to claim 2, wherein the compound of formula (III) is reacted with the compound of formula (IV) in a mole ratio ranging from about 1:1 to about 1:5.

4. The method according to claim 2, wherein the reaction medium is selected from an aqueous reaction medium and an organic reaction medium.

5. A method of preparing polymer, the method comprising polymerising under the control of a RAFT agent of formula (I) one or more ethylenically unsaturated monomers in a reaction medium selected from an aqueous reaction medium and an organic reaction medium:

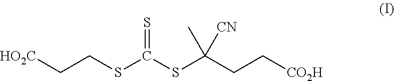 [SMILES: CC(C#N)(CCC(=O)O)SC(=S)SCCC(=O)O]

6. The method according to claim 5, wherein the one or more ethylenically unsaturated monomers comprises a monomer of formula (V): 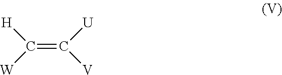
where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹, NR^{x}R^{y}, and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

7. The method according to claim 5, wherein the one or more ethylenically unsaturated monomers comprises a monomer of formula (Vb): 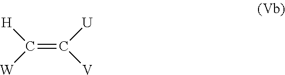
where W is H or forms together with V a lactone, anhydride or imide ring; U is selected from C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹ and SO₂R¹; and where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

8. The method according to claim 5, wherein
the polymer prepared has a dispersity (Ð) of less than 1.4.

9. The method according to claim 5, wherein the polymerisation is promoted thermally, photolytically or a combination thereof.

10. A method of preparing polymer, the method comprising:
(a) combining in a reaction medium a compound of formula (III), a compound of formula (IV) and one or more ethylenically unsaturated monomers; 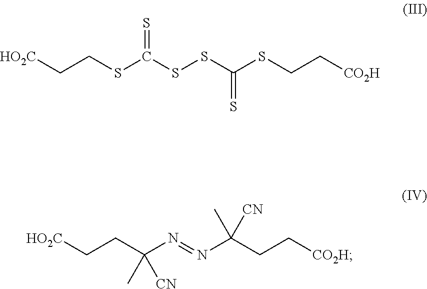 [SMILES: O=C(O)CCSC(=S)SSC(=S)SCCC(=O)O ; CC(C#N)(CCC(=O)O)/N=N/C(C)(C#N)CCC(=O)O]
(b) reacting the compound of formula (III) with the compound of formula (IV) to form a RAFT agent of formula (I); and 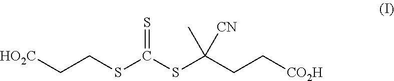 [SMILES: CC(C#N)(CCC(=O)O)SC(=S)SCCC(=O)O]
(c) polymerising in the reaction medium the one or more ethylenically unsaturated monomers under the control of the RAFT agent of formula (I).

11. The method according to claim 10, wherein the reaction medium is selected from an aqueous reaction medium and an organic reaction medium.

12. Polymer of formula (II): 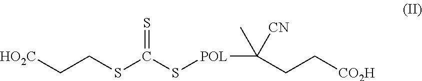
where POL is a polymer chain comprising the polymerised residues of one or more ethylenically unsaturated monomers.

13. The polymer according to claim 12, wherein POL is a polymer chain comprising the polymerised residues of one or more ethylenically unsaturated monomers of formula (V): 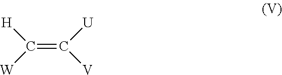
where W is H or forms together with V a lactone, anhydride or imide ring; U is selected from H, C₁-C₄ alkyl, CO₂R¹ and halogen; V forms together with W a lactone, anhydride or imide ring or is selected from optionally substituted aryl, alkenyl, CO₂H, CO₂R¹, COR¹, CN, CONH₂, CONHR¹, CONR¹₂, PO(OR¹)₂, PO(R¹)₂, PO(OH)R¹, PO(OH)₂, SO(OR¹), SO₂(OR¹), SOR¹, NR^{x}R^{y}, and SO₂R¹; where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain, and where R^{x} and R^{y} form together with N an optionally substituted heterocyclic or heteroaryl group.

14. The polymer according to claim 12 having a dispersity (Ð) of less than 1.4.

15. The polymer according to claim 12 in the form of a copolymer.

16. The polymer according to claim 13 having a dispersity (Ð) of less than 1.4.

17. The polymer according to claim 13 in the form of a copolymer.
